(19) United States
(12) Patent Application Publication
(10) Pub. No.: US 2006/0222723 A1
(43) Pub. Date: Oct. 5, 2006

(54) Terpene-based composition of substances, a method for its preparation and a method for its dispersal into the atmosphere

(76) Inventor: Matteo Bevilacqua, Padova (IT)

Correspondence Address: SUGHRUE MION, PLLC, 2100 PENNSYLVANIA AVENUE, N.W., SUITE 800, WASHINGTON, DC, 20037, US

(21) Appl. No.: 10/515,656

(22) PCT Filed: May 23, 2003
(86) PCT No.: PCT/EP03/05412

§ 371 Date: Mar. 3, 2006

(30) Foreign Application Priority Data
May 24, 2002 (IT) PD2002A000138

(51) Int. Cl. A61K 36/00

(52) U.S. Cl. 424/769

(57) ABSTRACT

A composition of terpene-based substances comprises a sesquiterpene fraction and/or a triterpene fraction derived from natural resins in which the fractions are intimately mixed with a gum component. The sesquiterpene and/or the triterpene fraction is present in the composition, separated from the gum component.

The present invention relates to a terpene-based composition of substances according to the preamble to the main claim, for applications in the hygiene and pharmaceutical fields. The invention is also directed towards a method for the preparation of the composition, as well as towards a method for its dispersal into the atmosphere.

The terpene-based substances used are preferably derived from natural resins and, in particular, from olibanum, myrrh and Dacryoides incense.

Olibanum, which is commonly known as incense, true incense, or frankincense (to be distinguished from the generic name “incense” which is used to indicate a mostly resinous substance used for its perfumes, which are generally produced by burning the substance) is a hardened gum resin which flows from incisions in the bark of various plants of the Boswellia species, of the Burseraceae family, which are mostly shrubs that grow sparsely in the savannah on calcareous, chalky and dry soils which are present in areas between the equator and the tropics, in particular in India, Somalia, Eritrea, Saudi Arabia and Yemen.

Olibanum is known for its balsamic and anti-fermentation properties and has been used from very early times in the treatment of ulcerous recto-colitis, parasitosis and eye diseases. In Chinese popular medicine in particular, it is used for the treatment of diseases of the respiratory system and of the stomach, and as ointment for external use, particularly for skin ulcers, burns and muscle pain.

The composition of olibanum varies according to the plant species which produce it. Chemical analysis has demonstrated the predominant presence of pentacyclic triterpenes with high molecular weights [such as beta-boswellic acids (C₃₀H₄₈O₃; molecular weight 456.7, melting point 228° C.), acetyl-beta-boswellic acid (C₃₂H₅₀O₄; M.W. 498.74; M.P. 225° C.), 11-keto-beta-boswellic acid (C₃₀H₄₆O₄; M.W. 470.69; M.P. 197° C.), and acetyl-11-keto-beta-boswellic acid (C₃₂H₄₈O₅; M.W. 512.73; M.P. 274° C.)]. Cyclic monoterpenes, bicyclic terpenes and sesquiterpenes are also present, but to a lesser extent (about 12% in Somalian incense), whereas the gum component is about 40% (Bevilacqua M. and Coll., 1997).

The triterpenic acids mentioned above are gaining ever greater importance owing to their cortisone-type anti-inflammatory action with inhibition of the enzyme 5-lipo-oxygenase and reduction of the production of leukotrienes (Safayhi H. and Coll., 1992, Kweifio-Okai G., 1992), in the treatment of particularly serious and widespread diseases in which there is a hyper-production of leukotrienes, such as bronchial asthma (in which these acids have also shown an anti-elastase activity similar to alpha-1-anti-trypsin), chronic polyarthritis (Sander O. and Coll., 1998), chronic colitis (Gupta and Coll., 2001), in Crohn's disease in the active phase (Gerhardt H. and Coll. 2001), and in experimental ileitis (Krieglstein C. F. and Coll., 2001). There are also indications for effective therapeutic use of triterpenic acids in the treatment of multiple sclerosis, which is comparable experimentally with autoimmune encephalomyelitis, in which boswellic acids are effective on the symptoms (Wildfeuer and Coll., 1998), and in inhibiting rejection in the same manner as high doses of cortisones (Dahmen U. and Coll., 2001).

Moreover, triterpenic acids perform an anti-tumour activity, for example, in leukaemia (Shao and Coll., 1998); in particular, boswellic acid has been used for the treatment of brain tumours (Simmet and Coll., 1999). The mechanism of their action is thought to be Inhibition of the synthesis of proteins, of RNA and, above all, of DNA (Huang M. T. and Coll., 2000).

Amongst the triterpenes of olibanum, oleanolic acid and ursolic acid have been identified; these are pentacyclic triterpenes which are of interest owing in particular to their anti-tumour activity, their cytotoxic, anti-mutagenic, anti-invasive and anti-angiogenic activity, and their activity in inducing apoptosis in tumour cells and in the prevention of malignant transformation of normal cells. It has been shown that ursolic acid also Interferes with numerous enzymes including those involved in DNA synthesis (Novotny L. and Coll., 2001), in particular, it is a catalytic inhibitor of human topoisomerase I and II alpha (Syrovets T. and Coll., 2000) with real therapeutic possibilities in skin tumours (Tokuda H. and Coll., 1986), in glioblastomas (Ciusani and Coll., 2002), in meningiomas (Roessler K. and Coll., 2002), in leukaemia (Simon A. and Coll., 1992), and probably in small-cell lung tumours, and in cancer of the colon (Kouniavsky G. and Coll., 2002) and of the breast (Hossain M. S. and Coll., 2002).

Pentacyclic triterpenes, in particular, ursolic acid, also have a clear antiviral activity, activity in inhibiting the Epstein-Barr virus (Tokuda H. and Coll. 1986), and activity in inhibiting HIV-I protease (Min B. S. and Coll., 1999). Diterpenes also have a synergic antiviral and anti-tumour action with one another, with inhibiting effects on the Epstein-Barr virus (Konishi T. and Coll., 1998), as well as anti-malarial activity.

The activity of terpenes, in particular of triterpenes, on DNA viruses such as the Epstein-Barr virus suggests that they might be active on the aetiological agents of neoplasia correlated therewith, such as nasopharyngeal carcinoma, Burkitt's lymphoma, Hodgkin's disease, and B-cell lymphoma.

Their activity on RNA viruses such as HIV suggest that that terpenes, in particular triterpenes, may also be active on the retrovirus family, particularly on the “oncogenic viruses” of the avian leukosis type (e.g. Rous sarcoma virus), mammal viruses of type C (e.g. Abelson leukaemia virus), of type B (mouse breast tumour virus), and of type D (Mason Pfizer monkey virus); on the “slow viruses” such as HIV 1 and 2 which cause AIDS, the Visna virus which causes lung diseases, and at the level of the central nervous system (SNC) in sheep, on the feline immunodeficiency virus which causes immunodeficiency in cats, and on the “foamy viruses” (monkey “foamy virus”). Myrrh is a fragrant, resinous substance produced mostly from various types of plant of the Commiphora species of the Burseraceae family, from which it exudes spontaneously, or through incisions in the bark. Myrrh hardens into drops or lumps in air, softens without melting at about 100° C., and melts at about 120° C.

The applications of myrrh in the medical field are many and ancient; it is known as a disinfectant (for wounds in poultice form, for the oral cavity in alcoholic solution), as an anti-microbial agent, insect-repellent, insecticide (Shonouda M. and Coll., 2000), and larvicide (Massoun A. M. and Labib I. M., 2000, Massoud A. M. and Labib I. M., 2001), and as an anti-inflammatory, and painkiller (Atta A. H and Alkofahi A., 1998).

Various studies have also shown the positive activity of myrrh against fungi (mycosis of the feet), termites, scabies mites (Bernadet M; 1983), schistosomes (Sheir Z. and Coll., 2001), flukes (Massoud A. and Coll., 2001), and amoebae (Sharma P. K. and Sharma J. D., 1996). Myrrh also has anti-spastic, anti-ulcer, cytoprotective (Al-Harbi M. M. and Coll., 1997), and healing activity, sedative activity on the central and autonomic nervous systems, probably attributable to sesquiterpenes such as furanoeudesma-1,3-diene, by a mechanism of interaction with the brain opioid receptors (Dolara P. and coll., 1996), local anaesthetic activity, attributed to sesquiterpenes (Dolara P. and Coll., 2000), hypocholesteraemizing and hypotriglyceridaemizing activity (Verma S. K. and Bordia A., 1988), immunostimulating activity (Delaveau P. and Coll., 1980), anti-tumour activity (Qureshi S. and Coll., 1993), anti-thrombotic activity (Olajide O. A., 1999), and anti-diabetic activity (al-Awadi F. and Coll., 1991).

According to traditional Chinese medicine, incense and myrrh, when used in combination with other compounds, are useful for the treatment of some types of tumours and for haemopoiesis, as well as for digestive disorders and for skin and muscle conditions.

The gum resin “Dacryoides incense”, which is practically unknown in the western world, was discovered and investigated only In very recent times (Bevilacqua M. and Coll., 1997). This gum resin, which has physical characteristics different from those of olibanum, is produced from plants of the Burseraceae family, Dacryoides genus, klaineana species, which are known by the Creole name Tghurai, and are present in subequatorial West Africa.

Dacryoides incense is substantially different from common Boswellia incense; it is characterized in that it has a low content of gum component (24%), softens without melting at about 80° C., melts at about 90° C., and is very slightly aromatic. It is composed predominantly of bicyclic terpenes and sesquiterpenes, with a percentage assessable as about 52%, about 18% of cyclic terpenic alcohols, about 0.5% of terpenic ketones, about 6% of oxyterpenes, partly from triterpenes, and is practically free of monoterpenes.

The first studies of Dacryoides incense found anti-microbial activity, in particular anti-mycotic activity against thermophilic actinomycetes (Bevelacqua and Coll., 1997), and anti-inflammatory and insect-repellent activity.

Olibanum, myrrh and Dacryoides resins, which are likely to be rich in triterpenes, as in the Cimicifuga spp. (Takahira M. and Coll., 1998), and diterpenes as in the Pinaceae, probably have anti-malarial activity.

As shown by the foregoing description, terpene-based substances, particularly derived from natural olibanum and myrrh resins, are used widely in the medical/hygiene field. Compositions of these resins combined with one another in various ways, as well as with propolis, are described, for example, in International patent application WO 97/02040 in the name of Michelin and Bevilacqua. However, although these combinations have improved anti-inflammatory and anti-microbial activity, they are not yet sufficiently effective and, moreover, their use is limited by the possibility of side effects arising owing to the presence of gum residues in the incense, or of wax and pollens in the propolis.

In the technical field in question, terpene-based substances derived from natural resins, for example, those described above, are prepared in various forms, according to the manner of use, that is: in alcoholic solution, dissolved in oil, emulsified in water, or even In micronized powder form.

However, the known methods for the preparation of these substances have some disadvantages which in fact limit both the overall effectiveness of the composition and its type of use.

A first disadvantage is the poor efficiency of the methods of separating the various components which are present in a natural resin of the type in question. As mentioned briefly above, the natural resins of interest are generally composed of a gum component, constituted mainly by rosin and sugars (pentose and hexose) in which the terpenic substances to be separated, which in turn can be grouped, for convenience of identification, into a monoterpene fraction, a sesquiterpene fraction, and a triterpene function, are intimately mixed and dispersed.

A first known separation technique provides for the stripping of the resins in a current of steam, possibly under slight pressure. This technique enables a good separation of the low boiling fraction, typically the monoterpene fraction (C₁₀), to be achieved but the heavier sesquiterpene and triterpene fractions remain substantially trapped in the resin.

A second known separation technique provides for extraction with solvent. However, the degree of separation achieved is unsatisfactory and this technique also normally involves degeneration, even though only partial, of the terpene fractions treated.

A second disadvantage encountered in the preparation of the known compositions arises during the grinding of the above-mentioned natural resins. This step is in fact hindered by the presence of the gum component which, owing to its rheological behaviour, tends to stick to the grinding-mill rotor-blades, preventing correct and functional grinding of the resins, particularly when a particle size of less than 5 microns is required for the finished product. One of the known methods for the use of the terpenic substances of the resins, prepared in one of the forms listed above, provides for its dispersal in the atmosphere by evaporation by heating at a controlled temperature, for example of about 100° C. However, this method is limited by the fact that dispersal is achieved to a satisfactory extent solely by the low-boiling, volatile fraction of the terpenic substances contained in the resins, whereas the heavier fractions remain in the resin. Moreover, this method is limited by the fact that an excessive rise in temperature may lead to degradation of the terpenic substances.

The main object of the present invention is to provide a novel composition of terpene-based substances derived from natural resins which has more effective medical/hygiene activity than known compositions.

Within the scope of this object, the present invention also proposes to establish a method for the preparation of the above-mentioned composition, which method overcomes the limitations of the methods described above with reference to the prior art.

A third object of the invention is to identify novel therapeutic and hygiene applications for the composition of terpene-based substances.

A fourth object of the invention is to provide a novel method for the dispersal of the composition into the atmosphere.

These and other objects which will become clearer from the following detailed description are achieved by the invention by means of a composition of terpene-based substances, a method for its preparation, as well as a method for its dispersal into the atmosphere, in accordance with the appended claims. In a first aspect of the invention, the composition of terpene-based substances according to the invention comprises a sesquiterpene fraction and/or a triterpene fraction, separated from the gum component in which they are intimately mixed in the starting natural resins. As the results of the tests given in the description below will show, a considerable strengthening of the therapeutic and hygiene properties of the substances in the natural state is thus achieved. In particular, this strengthening effect has been shown in relation to the sesquiterpene fraction derived from Dacryoides incense.

The composition is preferably obtained from natural resins by separating the monoterpene, sesquiterpene, and triterpene fractions of olibanum, myrrh and Dacryoides incense, respectively, from the gum component, and combining these fractions with one another or with other terpene-based substances, in a balanced manner, in dependence on the application of interest.

The method for the preparation of the composition of the invention from the natural resins indicated above provides for a step of separation of the various components of the resins (gum component and terpene fractions) followed by an optional step of grinding and combining of the desired components, in accordance with the methods and proportions preselected for the use indicated.

According to a principal characteristic of the present invention, the components are separated from one another by a stripping process of the above-mentioned resins, carried out in a dry gaseous stream at predetermined temperature levels.

The resin to be treated is washed beforehand with distilled water at ambient temperature to remove any impurities such as residues of earth or wood particles and is then placed in a column arranged for the stripping operation. The gaseous stream is preferably caused to flow downwards from above and the gas used may advantageously be dehumidified air, the residual moisture content of which is no more than 100 ppm and preferably between 50 and 100 ppm.

A first stripping step takes place with a gaseous stream at a temperature of between 95° C. and 105° C. with a flow-rate preferably of between 0.25 and 0.30 m/sec.

During this step, the predominantly monoterpene fraction (C₁₀) is extracted from the resin treated and is recovered downstream of the column by cooling of the gaseous stream. The monoterpene fraction thus obtained is in gel form. Upon completion of this first stripping step, which generally lasts for a period variable between 15 and 25 minutes depending on the type of resin, the gaseous stream is brought to a temperature of between 130° C. and 138° C., preferably to a temperature of 135° C., and is kept in these conditions for a further 15-25 minutes.

At this temperature, melting of the lowest-melting portion of the sesquiterpene fraction (C₁₅) is observed and this portion is separated from the resin by pouring. The temperature is then brought to a value between 138° C. and 142° C., preferably to 140.6° C. at which, during a period of time similar to the previous one, the gum component is stripped by evaporation (as heavy gas) and is partially recovered.

The temperature of the gaseous stream is then increased to a value of between 180° C. and 200° C., preferably 190° C., and maintained for a period of 15-25 minutes, so as to bring about separation by pouring also of the remaining portion of the sesquiterpene fraction. The resin component which remains in the column is constituted substantially by the triterpene fraction (C₃₀), the melting point of which is above 190° C.

Upon completion of the process described, substantially the following components are obtained, separated from one another: a monoterpene fraction, a sesquiterpene fraction, a triterpene fraction, and a gum component. The fractions of each of the resins indicated above, thus separated, can be combined with one another in various ways and associated with one or more terpene fractions obtained from the other resins in question, once they have been subjected to the same separation process.

The sesquiterpene and triterpene fractions (obtained in solid form by the stripping treatment) may also, if required, be subjected to fine grinding, for example, by means of a Nietsche-Condux pulverizing mill, to give powders with an average particle-size of about 5 microns, or to superfine grinding, for example, by means of a Hosokawa pulverizing mill, to give powders with an average particle size of about 0.8 microns.

It will be appreciated that this grinding operation can be performed and controlled without any technical difficulty since the terpene fractions are free of gum component. It is thus also possible to reach a degree of pulverization which would not otherwise be achievable, in the presence of the gum component.

The method given above also permits a more accurate separation of the components of each resin treated. The gaseous stream can in fact be brought from the temperature of 95-105° C. to temperatures even greater than 200° C. by successive temperature increases of 5° C. The period for which the air flows through the column upon completion of each temperature increase may vary from the 15-25 minutes of the standard steps, according to the substances to be separated. It is thus possible to achieve a more precise subdivision of the terpene compounds contained in the natural resins; in particular, it is possible to separate, in addition to monoterpenes (C₁₀), sesquiterpenes (C₁₅), and triterpenes (C₃₀), also diterpenes (C₂₀), tetraterpenes (C₄₀), and polyterpenes ([C5]n where n>10 with carbon-carbon (C—C) bonds with the cis configuration).

The above-described method for the separation of the terpene fractions enables pharmaceutical products comprising the compositions of the invention to be prepared in various forms and by various methods in dependence on the specific application desired.

A first type of preparation provides for the preparation of a pharmaceutical product in powder form, comprising the terpene fractions of the olibanum, myrrh and Dacryoides resins in the ratios specified below, as well as the respective gum components, in addition to a pharmaceutically acceptable vehicle such as polyethylene glycol/hydrogenated castor oil, for example Cremophor®™ RH40 produced by BASF, and similar products.

This type of preparation, in which the gum component is also included, (resin in toto), may be useful when it is intended to promote a greater local effect by slowing down the absorption of the active ingredients (constituted by the terpene fractions), for example, in topical skin applications, or in oral administration when it is of advantage to increase the intestinal transit time to achieve a longer time spent by the active ingredients transported, in the regions in which their pharmacological effects are advantageous.

A second type of preparation provides for the preparation of a pharmaceutical product in powder form, similar to the previous one but without the gum components of the resins treated.

This type of preparation, particularly when ground uniformly to fine or superfine level, offers the advantage of greater solubility and optimal suitability for aerial use, as well as considerably reducing the possibility of side effects. For example, in medicine, the powder with particles of about 5 microns can be administered into the upper airways by inhalation; the powder, micronized to below 5 microns, down to 0.8 microns, offers the further possibility of reaching the whole lung, down to the alveoli, without risk of pneumoconiosis, given that it is free of the gum component which is poorly soluble; for local application to the skin and to the mucous membranes, it may reduce the risk of contact allergy (also resulting from the gum component).

A third type of preparation provides for the preparation of a pharmaceutical product in the form of essential oil in which one or more of the terpene fractions, separated and pulverized In accordance with the method described above and suitably combined in accordance with the therapeutic indication desired, is eluted with ethanol in various dilutions, or in solution with vegetable oils (preferably with polyunsaturated omega-3 fatty acids or with linseed oil) or even with essential oils of other substances (for example, hyssop essential oil) as described below.

In a first preferred method for the preparation of the essential oils of the composition according to the invention, the terpene fractions are eluted in ethanol with a dilution of from 5% to 15%, that is, to form a dense liquid, almost “resin honey”, for use for dispersal in the atmosphere in accordance with the method described below, or for oral applications by spray or by mouth, or even for topical applications as ointment, salve or cream.

For oral applications, a preferred embodiment provides for the preparation of the composition of the invention in capsules, the shells of which are made of water-soluble material, for example, with polylactic acid. The active ingredients are thus released directly into the gastrointestinal system, avoiding contact with the oral cavity (an effect which is particularly useful in the presence of substances having an unpleasant taste).

In a second method for the preparation of the essential oils of the composition according to the invention, the terpene fractions of olibanum, of myrrh, and of Dacryoides incense, with or without gum content, are eluted in oily or alcoholic solution with a dilution of from 15% to 40%. This enables a more or less viscous mixture to be obtained, according to need, which, when applied to a surface, forms a transparent, dry, impermeable and non-adhesive film which remains unchanged for at least 24 hours, with very extensive applicational possibilities (for example, it may be applied as an ointment or spray to decubitus ulcers, or to skin ulcers, or to internal ulcers, or to stoma sites such as tracheotomies and colostomies); moreover, it enables high and uniform concentrations to be achieved on the part to be treated, keeping the transfer rate stable and avoiding discontinuous treatments.

As a result, the product applied to the skin locally may not only have a local action with prolonged and constant effect but may also act as a “transdermal patch” since the active ingredients are liposoluble and are absorbed into the organism (for example, into the lungs and into the intestine), avoiding first-pass gastrointestinal and hepatic metabolism. Moreover, in comparison with transdermal patches, any side effects due to the constituents of the patch, further cost, or difficulties in supply or application, are avoided.

Naturally, it is also possible to apply the composition prepared in essential-oil form to patches, thus forming a true transdermal patch. In a preferred embodiment, the structure of the patch may be formed of fibres of water-soluble material, for example, polylactic acid. If the thickness of the structure of the patch is of suitable dimensions, simultaneous absorption of the composition into the organism and decomposition of the structure of the patch over a predetermined period of time can thus advantageously be achieved.

A fourth method for the preparation of the composition of the invention provides for the dispersal of the terpene fractions derived from the myrrh, olibanum and Dacryoides resins and free of the respective gum component, in water, in emulsion form. These fractions are preferably ground to superfine level when they are to be vaporized for administration to the lungs or sprayed onto stoma sites.

The composition of the invention, though giving a synergic effect substantially at any ratio between the fractions, gave the best results when the terpene fractions contained therein were suitably balanced; in particular, when the monoterpene fraction, the sesquiterpene fraction, and the triterpene fraction were present in an optimum ratio by weight of 1:1:1 with a variation of +0.2 for each fraction.

Naturally, to achieve this balance, the quantities of resin to be treated will depend on the variety of plant from which the resin has been obtained.

The following table gives indicative percentage values of each terpene fraction, relative to the total terpenic substances present in the natural resins listed below:

| | Boswellia | Boswellia | Commiphora | Dacryoides |
|---|---|---|---|---|
| | carteri | sacra | molmol | klaineana |
| | olibanum | olibanum | myrrh | incense |
| Monoterpenes | 25 | 40 | 10 | 5 |
| Sesquiterpenes | 20 | 20 | 45 | 75 |
| Triterpenes | 55 | 40 | 45 | 20 |

For particular applications, as will become clearer from the examples given below, the composition of the present invention may advantageously be enriched with specific essential oils, in particular, with hyssop essential oils and/or with polyunsaturated omega-3 fatty acids.

Hyssop essential oil is known for its anti-inflammatory, anti-microbial (antiviral, bactericidal, fungicidal, anti-parasitic), anti-bronchospastic, painkilling, and vasoprotective properties; it is mentioned in the British Herbal Pharmacopoeia as a remedy for bronchitis and colds; it is used in aromatherapy against influenza, coughs and expectoration, laryngitis and tracheitis, gastralgia, hepatitis, bruises, and acne rosacea (Bernadet M., 1983). It has recently been found to have activity against mites such as house-dust mites, Dermatophagoides pteronissimus, Tyrophagus putrescentiae, Sarcoptes scabiei, and Tetranychidae (Kawada H., 1999) and as an insect-repellent and insecticide, for example, against Musca domestica and insects that are harmful in agriculture. Hyssop essential oil is used in combination with myrrh and Incense in the formulation of cosmetic and dermatological compositions.

The hyssop essential oil which is preferably used is derived from Hyssopus officinalis, decumbens variety; this oil is rich in monoterpenes (about 80% of the terpene-based substances, in particular 60% is trans-linalol-oxide) and, to a lesser extent, in sesquiterpenes (about 20% of the terpene-based substances) whereas the triterpene fraction, as well as the neurotoxic ketonic fraction is practically absent.

Polyunsaturated omega-3 fatty acids, better known as “fish oils” are known for performing a prominent role in the modulation of cell functions and in the response to external stimuli. They are known for their anti-phlogogenic activity and are considered to have a particular anti-Helicobacter pylori action and an inhibitory action on topoisomerase (Suzuki K. and Coll., 2000). The polyunsaturated omega-3 fatty acids which are preferably used are those with a content of EPA (eicosapentanoic acid) and DHA (docosapentanoic acid) of more than 95%. For use in compositions for topical use, owing to their unpleasant odour, they may advantageously be replaced with linseed oil which contains about 53% of linoleic acid from which alpha-linoleic acid, parent of the polyunsaturated omega-3 fatty acids, is derived.

On the basis of the composition proposed above, whether or not it is enriched with hyssop and/or omega-3 fatty acids, it is possible to prepare pharmaceutical products, combined in various ways according to their purposes, which may be in the form of tablets, pastilles, capsules, pills, sprays, sweets, chewing gum, liniments and throat pastilles, toothpastes, mouthwashes, gargles, or oils, solutions, emulsions, ointments, salves, creams, powders with various degrees of micronization for various uses as preparations for inhalation or nebulization, or sprays, aerosols, suppositories, globules, poultices, transdermal patches, and even aromatherapy preparations, and for cosmetic uses as fixatives and fragrance components in soaps, detergents, cosmetics, perfumes, and face powders.

The therapeutic and hygiene properties of the terpene-based substances of the present invention, both separately and in combination with one another, have been tested by means of a series of tests directed towards testing their anti-microbial activities against various collection microbial strains (Gram+, Gram−, fungi). At the same time, they were compared with those of the three resins, tested individually, and with those of other known natural substances such as thyme, oregano, nutmeg, basil, helychrisum, myrtle, tea-tree, and eucalyptus radiata.

All of the substances tested were used In the form of essential oils and were tested with the following microbial strains: Escherichia coli ATCC 8739, Enterococcus hirae ATCC 10541, Pseudomonas aeruginosa ATCC 15442, Staphylococcus aureus ATCC 6538p, Klebsiella pneumoniae ATCC 13882, Serratia marcescens ATCC 8100, Candida albicans ATCC 10231, Aspergillus niger ATCC 16404, Proteus mirabilis ATCC 43071.

The tests were carried out by following the “Broth microdilution assay” method according to Hammer K. A. and Coll., 1999, approved by the NCCLS, applied to essential oils (the method consists In the preparation of a liquid culture broth both for the dilutions of the bacterial strain and for the dilutions of the substances to be tested).

The substances which showed significant results for each bacterium, with dilution at least to 1:160, are given below:

Klebsielle pneumoniae: Dacryoides incense, olibanum, hyssop, oregano, nutmeg, basil, myrtle, eucalyptus radiata

Proteus marabilis: Dacryoides incense, oregano, basil, tea-tree,

Serratia marcescens: Dacryoides incense, thyme, oregano, basil, tea-tree, eucalyptus radiata,

Enterococcus hirae: Dacryoides incense, oregano,

Escherichia coli: Dacryoides incense, hyssop, oregano, myrtle,

Staphylococcus aureus: Dacryoides incense, hyssop, oregano,

Candida albicans: Dacryoides incense, olibanum, hyssop, myrrh, oregano, basil, myrtle,

Pseudomonas aeruginosa: no substance was active.

For all of the bacterial strains indicated, in relation to which the individual substances were found active, a series of second compositions of the invention in which the terpene fractions (mono-, sesqui- and tri-) derived from Dacryoides, olibanum and myrrh were associated in different ratios were also tested. In all of the cases tested, an improvement in the anti-microbial activity of at least one further dilution was found. The best results have been obtained with composition being substantially in a 1:1:1 ratio with a variation of ±0.2.

The improvement was even more marked in all of the cases tested with at least two further dilutions when the essential oils solely of the sesquiterpene fractions, both of Dacryoides and of the resins, were used in combination with the respective monoterpene fractions.

A second series of tests relating to the anti-microbial activity of the substances of the composition of the invention was carried out specifically in relation to the bacterium Helicobacter pylori NCTC G-21, with regard to which there do not seem to be any references in the literature to activity exhibited by the three resins, either individually or in combination with one another.

The tests were carried out by a different method (seeding in BHI and subsequent inoculation on plates, Kirby-Bauer method) on the basis of essential oils of olibanum, Dacryoides and myrrh, evaluated individually and in combination with one another. The composition of the Invention was also evaluated with the further addition of hyssop essential oil (which was also tested individually) and of omega-3 fatty acids. For comparison, essential oils of thyme, oregano, nutmeg, basil and helichrysum were also tested.

The substances tested individually, when diluted in 1:10 NB, showed the following anti-Helicobacter activity, given in decreasing order of efficacy with reference to the measurement of the diameter of the inhibition halo: Dacryoides incense (45 mm), myrrh (40 mm), olibanum (30 mm), hyssop (22.5 mm), helichrysum (22 mm), basil (20 mm), nutmeg (16 mm), thyme (13 mm) oregano (9 mm), negative control with alcohol and with olive, maize and linseed vegetable oils. The anti-Helicobacter activity was clear, even at a dilution of 1:100.

The test relating to the composition of Dacryoides incense, olibanum and myrrh in the ratios indicated above surprisingly showed an anti-Helicobacter effect which was greater than that obtained with the individual resins, and was quantifiable in at least one further dilution. This proves the clear presence of a synergic effect between the resins, which effect is clearer when the above-mentioned ratios between the terpene fractions are adhered to.

To complete the investigation into the anti-Helicobacter activity of the three resins tested, some tests were carried out with the use solely of specific terpene fractions thereof. These tests showed that a first composition formed by the sesquiterpene fraction derived from Dacryoides incense and by the monoterpene fraction of olibanum, as well as a second composition formed by the sesquiterpene fraction derived from Dacryoides incense and by the sesquiterpene fraction of myrrh and/or of olibanum had an even more marked anti-Helicobacter activity; in fact the same inhibition diameter was obtained even at twice the dilution, both with Dacryoides Incense and olibanum monoterpenes and with the combination of the sesquiterpenes of the three resins.

Further tests were carried out to check the combination of the three resins or of some of their terpene fractions with hyssop (in the form of essential oil) and with polyunsaturated omega-3 fatty acids.

For combination with hyssop, the best results (average inhibition diameter of more than 50 mm) were obtained with compositions in which the ratio between olibanum from Boswellia sacra, myrrh, Dacryoides and hyssop was 1:1:0.5:0.5 with a variation of ±0.2 for each component.

For combination with polyunsaturated omega-3 fatty acids, the best results were found with compositions in which the ratio between the three resins in toto, on the one hand, and the omega-3 acids, on the other hand, was 1:10±4. Here again, the combination showed a anti-Helicobacter effect which was greater than that obtained with the individual resins, and which was quantifiable in at least one further dilution.

The discovery of the anti-Helicobacter effect both of the three resins and of hyssop variety decumbens has considerable clinical importance, not only because of its more intense anti-microbial activity demonstrated in comparison with the other known substances but, above all, owing to the absence of side effects which, in contrast, are exhibited with the use of the other substances (particularly owing to probable neurotoxicity of the latter, probably due to the presence of ketones, as in helichrysum and in other hyssop varieties).

A third series of tests carried out by the method given above evaluated the anti-microbial activity of the composition of the invention with respect to the bacterium Campylobacter jejuni (a wild strain isolated from a chicken farm).

As in the previous case, essential oils of olibanum, Dacryoides incense, myrrh and hyssop were tested individually and in combination with one another. Moreover, the substances were tested, in powdered form, again in toto, and thus comprising all of the various terpene fractions, as well as the gum component.

The best results of the tests showed considerably greater anti-microbial activity of the composition with terpene fractions which were balanced in accordance with the 1:1:1 ratio than of the substances tested individually or with other ratios, achieving the same effects with an almost halved concentration of the substance.

For the substances in toto, the tests also showed good microbial activity when the substances were used unchanged, that is, without any dilution. This activity was wholly similar to that of disinfectants available on the market.

The anti-Campylobacter jejuni activity shown by the three resins and, in particular, by their combination in the preferred ratios by weight, has considerable importance in the zootechnical field since it is known that this bacterium causes high mortality in farm chickens, causing enormous economic loss. Moreover, it should not be forgotten that the infection caused in chickens by Campylobacter jejuni may subsequently be transmitted to man.

A final series of tests was carried out to check the activity of the substances described above against mites.

Hyssop essential oil, even with aerial administration, has recently been found to have activity against various species of mites, including house-dust mites such as Dermatophagoides pteronissimus (Kawada H., 1999), which is responsible for allergy, particularly of the respiratory system, of which the type most to be feared is bronchial asthma which affects about 5% of the general population; myrrh is also known to have some efficacy (Bernadet M., 1983) against scabies mites.

Olibanum, Dacryoides incense, and myrrh resins were therefore tested against Dermatophagoides pteronissimus and against common chicken mite (Dermanyssus gallinae) which has the advantage of being visible to the naked eye and hence more suitable for measurements.

The test consisted of the nebulization of the three micronized resins in a transparent balloon and the vaporization of hyssop essential oil (by means of the electro-emanator which is the subject of Italian patent No. 1287235), the admission of a uniform load of mites into the balloon, and two optical microscope readings (average count of mobile Dermatophagoides and their ratio to the immobile Dermatophagoides in four visual fields) after exposure for three hours and for six hours.

The substances were tested first of all separately and then in combination with one another.

Surprisingly, Dacryoides incense, tested individually was shown to be substantially as active as hyssop with a reduction of more than 30% in the number of living mites per field.

With regard to the activity of the various combinations of the resins and of their terpene fractions, a great strengthening of the anti-mite activity was found in a composition of Dacryoides incense and myrrh in toto with hyssop. This strengthening was even clearer with the use solely of the sesquiterpene fractions of Dacryoides incense and of myrrh in combination with hyssop, in equal proportions by weight.

A further series of tests was carried out to check the activity of the terpene-based substances derived from olibanum, myrrh and Dacryoides incense resins against retroviruses, particularly HIV, by testing them with tests of inhibition of viral replication in vitro. The preliminary results obtained would confirm the antiviral activity indicated in the literature and also showed the synergism of the action of the three substances in combination, in toto, as well as particularly of the sesquiterpene fractions derived mostly from Dacryoides incense, and the triterpene fractions.

The tests described above show a surprising synergic effect of the terpene fractions derived from olibanum, myrrh and Dacryoides incense resins.

Of particular importance and interest was the effect obtained by the combination of the sesquiterpene fraction derived from Dacryoides incense with the terpene fractions of the other resins. The following were strengthened in particular by this combination:

the anti-microbial action of the monoterpene fraction of olibanum,

the anti-microbial and anti-inflammatory action of the sesquiterpene fractions of myrrh and/or olibanum,

the painkilling effect of the sesquiterpene fraction of myrrh, and

the anti-inflammatory action of the triterpene fraction of olibanum and/or myrrh.

The extent of the synergic effect of the sesquiterpene fraction (particularly present in Dacryoides incense) with the other terpene fractions is so clear (anti-inflammatory and anti-microbial responses increased or even doubled) as to suggest the presence of novel terpene compounds.

It was also observed that this synergic effect was quite marked when the gum component was substantially absent or in any case limited to an overall percentage no greater than 2%. In this connection, it should be noted that, by means of the above-described method for the separation of the terpene fractions from myrrh, olibanum and Dacryoides resins, it is particularly easy to obtain the desired terpene composition free of gum component.

For a fuller understanding of the surprising properties described above, the terpene-based substances of the composition and in particular of Dacryoides incense, were subjected to deeper and detailed qualitative and quantitative analysis.

This analysis, which was carried out by GC/MS, GC/FID, GCCP and TGA techniques, disclosed the presence in the incense derived from Dacryoides klaineana of:

boswellic acids (predominantly beta-boswellic acid and 11-keto-beta-boswellic acid),

furan-eudesma-1,3,diene sesquiterpene (C₁₅H₁₈O, MW 214) in a quantity comparable to that found in myrrh, which explains the painkilling activity of Dacryoides incense,

tropolone, or beta-thujaplicin (C₇H₆O₂, MW 122) which has recognized bactericidal and fungicidal activity (Baya M. and Coll., 2001) by Inhibition of tyrosinase fungus (Shiino M. and Coll., 2001), against Schistosoma cruzi and against Plasmodium falciparum (Ren H. and Coll., 2001), such as also to suggest a novel class of anti-parasitic drugs with improved biological and pharmacological properties, as well as cytotoxic and anti-tumour activity (Matsumura E. and Coll., 2001) as inhibitor of ribonucleotide reductase (Tambualin-Thumin and Coll. 2001),

2-tert-butyl-1,4 naphthoquinone, which has antioxidant properties on 5-lipoxygenase (Worm G., 1994), chemo-preventive anti-tumour properties (Itoigawa M. and Coll., 2001), and probably anti-malarial properties (Kapadia G. J., and Coll., 2001),

2,3,5,6,3′,5′,6′-octamethyl [2,2] paracyclophane, (C₂₄H₃₂, MW 320) which is known for its anti-DNA virus and anti-RNA virus properties. In particular, various investigations are in progress on the possibility of the use of this compound in treatment against AIDS.

Besides these substances, the following sesquiterpene derivatives were also identified in Dacryoides incense:

ascissic acid (C₁₅H₂₀O₄, MW 262), a natural component of vitamin A known to exert hormonal action on the growth and development of plants, (Rapparini F., and Coll., 2001). This compound also is known to have anti-tumour activity since it inhibits the development of chemically induced tumours, and stops cell multiplication, probably by reduction of the growth hormone found in the tumour tissue. This anti-tumour effect is free of the toxicity typically associated with chemotherapeutics,

phenol, 4,4′-(1-methylethylidene)bis-(C₁₅H₁₆O₂, MW 228.29)

1-(4-methoxyphenyl)-2-phenyl ethane (C₁₅H₁₆O, MW 219.29)

oxacyclohexadecan-2-one (C₁₅H₂₆O₂, MW 240.38)

6-pentadecanone (C₁₅H₃₀O, MW 226.40)

nonyl phenol (C₁₅H₂₄O, MW 220.35)

benzaldehyde, 4-(octyloxy)-(C₁₅H₂₂O₂, MW 234.33)

p-tert-butylpivalophenone (C₁₅H₂₂O, MW 218.33)

octanal, 2-(phenylmethylene)-(C₁₅H₂₀O, MW 216.32)

1,6,10-dodecantrien-3-ol, 3,7,11 trimethyl-, [S-(Z)](C₁₅H₂₆O, MW 222.37)

cyclopentadecanone (C₁₅H₂₈O, MW 224.38).

All of the sesquiterpene derivatives listed above have in their molecular structure a plurality of reaction centres which render the entire compound unstable and chemically very reactive. It is therefore reasonable to suppose that these substances may easily react with one another or with other terpene elements, giving rise to novel compounds in the form of polymers or of oligomers (monoterpene+sesquiterpene+triterpene; monoterpene+sesquiterpene; sesquiterpene+triterpene) which would explain the surprising activity of the sesquiterpene fraction of Dacryoides incense when released from the gum component. In the natural resins, these novel compounds would not be able to form since the individual terpene elements in question are dispersed in a matrix constituted by the gum component. The intimate mixture with the gum component leads to substantial occupation of the reaction centres of the derivatives in question and consequently to their non-availability for combination with other reactive sites.

Within the natural resin, these elements are therefore in a “frozen” situation from which they are liberated as a result of the separation from the gum component.

The discovery of ascissic acid, as well as of tropolone, of 2,3,5,6,3′,5′,6′-octamethyl [2.2] paracyclophane, and of 2-tert-butyl-1,4 naphthoquinone in Dacryoides incense opens the way to its use in the treatment of tumorous diseases in general, at least as an adjuvant, so that a strengthening of the anti-tumour activity of the sesquiterpenes and triterpenes which are also present in the other two resins can be predicted. The results of some preliminary investigations carried out in mice with Erlich induced solid tumours confirm these theoretical assumptions.

The presence of tropolone, of the naphthoquinone compound, as well as of 2,3,5,6,3′,5′,6′-octamethyl [2.2] paracyclophane clearly indicates the possibility of using Dacryoides incense as an antiviral agent against DNA viruses and retroviruses, in particular also against the AIDS virus, making it also reasonable to assume a strengthening of the antiviral and anti-malarial action of the sesquiterpenes and of the triterpenes present in Dacryoides incense and/or in olibanum and in myrrh.

To assess the possibility of aerial administration of the terpene-based substances of the invention, olibanum (both Boswellia sacra and Boswellia carteri), myrrh and Dacryoides incense resins were also subjected to analysis of their volatility, suitably compared with that of other substances known in the technical field in question, such as hyssop, helichrysum and propolis.

Volatility was evaluated by heating to 100° C. for a prolonged period of time (longer than 7 days) with an electro-emanator with controlled temperature (for example, the electro-emanator of Italian patent No. 1287235), after which samples were taken by solid-phase micro-extraction (SPME).

The most favourable results were obtained with hyssop (more than 90% volatile over 72 hours), Boswellia sacra olibanum (70% volatile in 48 hours), and Boswellia carteri olibanum (35% volatile in 48 hours); the other substances tested were much less volatile: myrrh and Dacryoides incense (approximately 10% volatile in 72 hours), propolis and helichrysum, (10% volatile in 24 hours). The volatile fraction in the first 48 hours of heating was constituted substantially by monoterpenes (in olibanum, predominantly by alpha-pinene, limonene, myrcene, sabinene, beta-pinene, para-cymene). After heating for 48 hours, in all of the substances tested, the volatile fraction was considerably reduced, from 30% to 7% relative to the initial quantity, but remained for a prolonged time, more than 5 days, and was constituted principally by sesquiterpenes (predominantly beta-caryophyllene).

In view of the optimal volatility demonstrated by olibanum and by hyssop in comparison with the other substances, their anti-microbial activity on the same bacterial strains of Staphylococcus aureus and Escherichia coli as were used in the tests described above was evaluated.

For this purpose, a novel method for the analysis of the anti-microbial activity of volatile substances was established; the method provides for the following steps:

preparing a starting bacterial suspension of predetermined titre, for example 10⁸ UFC/ml,

titrating the suspension, for example by seeding 100 μl of the 10⁻⁵ and 10⁻⁶ dilutions on BHA,

distributing 50 μl of the suspension on a sterile watch glass and placing it to dry in a ventilated oven at a temperature such as not to kill all of the bacteria present in the suspension, for example, at 37° C. for 45-60 minutes (providing one glass for the control and one for the test, for each bacterial strain to be tested),

providing an electro-emanator with controlled temperature, heated to an analysis temperature for the substance (the temperature at which it is possible to appreciate even partial volatility of the substance or of a fraction thereof); for the substances of the invention, the analysis was performed at about 100° C. and preferably with the use of an electro-emanator of the type described in Italian patent No. 1287235,

depositing the substance to be analyzed on the electro-emanator and leaving it to heat for 5 minutes,

after drying of the bacterial suspension, bringing the glass to ambient temperature and placing it above the electro-emanator,

after a predetermined contact time (1-5 minutes) taking the glass and putting it in contact with the culture liquid collected in a flask (8.5 g of NaCl, 5 ml of tween 80 per 1 litre of milliQ water, sterilized in an autoclave),

filtering through filtering membrane (diameter 0.45 μm, Millipore) and placing on a petri dish of BHA,

after incubation for 24-48 h at 37° C., counting the bacterial UFCs present on the surface of the filtering membrane.

After exposure to the vapours both of olibanum and of hyssop for 1-5 minutes, a significant reduction of more than 50% in the bacterial colonies was recorded, which justified the use of these substances in aromatherapy and could also justify its use in the treatment of phlogistic and infective conditions of the respiratory system.

To increase the possibility of the aerial use of the terpene-based substances of the solution, a method for the dispersal of these substances in the atmosphere has been established and permits the release into the air, not only of the low boiling components, as in the examples indicated above, but also of the heavier sesquiterpene and triterpene fractions, without leading to the damage which results from excessive raising of the temperature.

The method provides for the adsorption, on a porous material, of a mixture of the terpene-based substance to be dispersed, in a low-boiling liquid, and subsequent heating of the mixture.

According to a preferred embodiment of the method, the terpene-based substances, in the form of powders ground to superfine level, are eluted in ethanol with a dilution of 5-15% and put in contact with Syloid silica gel (amorphous type), in powder form or in flakes, having an average pore size of from 0.1 to 1 micron, cell dimensions of from 1 to 10 microns, and an adsorption capacity of at least 50%, preferably 80%.

The porous material used may also be constituted by other polymers such as polypropylene or polylactic acid having a suitable adsorption capacity.

The temperature of the mixture is then brought to a value of between 80° C. and 90° C. At this temperature, evaporation of the ethanol, which entrains the particles of the terpene substances with it, dispersing them in the air, is observed.

The best results are obtained with a ratio of silica-gel:eluate such that the entire resin-ethanol mixture is adsorbed in the silica gel, making maximum use of its adsorption capacity. If this capacity is 80%, this ratio is about 1.25:1.

As well as adsorbing up to 80% of the eluate of the resin in ethanol, substances of this type have been found to be an optimal vehicle since they permit a release into the air of more than 40% of the total resinous substance, not only of monoterpenes, as with the use of normal vaporization systems used up to now, but also of considerable portions of the sesquiterpene and triterpene fractions.

For the method described above to work correctly, the resin must have a very small particle size, for example, less than 2 microns and preferably about 0.8 microns, so that it can easily be entrained towards the outside atmosphere by the ethanol vapour. It should be noted that the degree of dispersal of each substance in the atmosphere is thus rendered substantially independent of its volatility.

With the dispersal method just described, the substances transported are released into the air over a controlled period of time and always in the quantity relating to the temperature to which the polymeric structure is heated. Moreover, at this temperature, the terpene-based substances do not undergo any alteration due to the heat, even when they are subjected to heating for many days. In contrast, such undesired physical/chemical alterations do take place when they are subjected to heating to temperatures greater than 100° C. even for a few hours, as in known dispersal methods.

The present method also overcomes the disadvantages encountered in known dispersal methods based on burning of the resins. It is in fact known that the products of this burning (incense fumes) may be particularly detrimental to human health; for example, they are irritants to the respiratory tract and may cause bronchospasm, particularly in the early years of life (Hong C. Y. and Coll., 1994); moreover, an increase in tumours has been found, particularly in the new-born, due to incense fumes inhaled by mothers during pregnancy (Preston-Martin S. and Coll., 1982).

The above-described method of dispersal into the atmosphere could have important applications in medicine for the treatment of bronchial asthma and of broncho-pneumopathies, in apiculture, for the prophylaxis and control of American Bacillus bee-larva pest, and for the sanitizing of confined spaces.

The composition of terpene-based substances of the invention could be usable in therapeutic applications both in man and in animals, owing to its anti-inflammatory, anti-mycotic, antibacterial, antiviral, anti-tumour, insect-repellent and insecticidal, anti-malarial, painkilling, vasoprotective, healing, and eutrophic activity. Moreover, it can usefully be used in cosmetics, both for perfuming cosmetic products and because of the preserving effect of the substances due to their anti-microbial power which is much greater than that of other substances containing single or few terpenes (predominantly monoterpenes).

In medicine, the therapeutic indications could be all of those in which pathological conditions of various aetiologies with inflammatory, microbial, or dystrophic components occur, post-traumatic and non-post-traumatic pathological conditions, as main or secondary events, conditions which are acute, chronic, in remission, or with effusion, and also conditions which are resistant to normal steroid treatment and to FANS, in circulatory changes, in some metabolic disturbances, and in tumours. These indications, distinguished by the method of administration, might be summarized as follows:

a) for topical use: myositis, arthrophathy, tendonitis, dermatitis and the like (seborrheic dermatitis, psoriasis, ichthyosis, occupational eczema, mycosis, herpes zoster), bruises, sores, ulcers and rhagades, capillary fragility and haematoma, acne rosacea, phlogopathy; for percutaneous treatment of sinusitis, broncho-pneumopathy, asthma, inflammation of the kidneys and of the urinary tract, phlogosis of the male and female genital tracts (menstrual abdominal pains, vulvovaginitis, salpingitis);

b) oral administration: oral hygiene, diseases of the mouth and of the teeth (gingivitis, pulpitis, periodontitis, tonsillitis), otitis, pathological conditions of the digestive system: conditions due to gastrooesophageal reflux, gastralgia, gastritis and duodenitis, in particular due to Helicobacter pylori, cholecystitis, enteritis, Crohn's disease, diverticulitis, haemorrhoids, inflammations of the kidney and of the urinary tract; diseases of the central and peripheral nervous systems of inflammatory, microbial, dystrophic and neoplastic aetiology, in particular anxiety and depression, epilepsy, schizophrenia, movement disturbances (Parkinsons disease, induced by drugs, progressive paralysis); phlogistic and demyelinising degenerative diseases (multiple sclerosis, Alzheimer disease, diseases caused by prions), upper and lower motor neuron diseases (lateral amyotrophic sclerosis, spinal muscular atrophy), use of and dependency from drugs (derivatives of opium, sedatives and hypnotics, cannabis and cocaine), tumour (glioblastoma, astrocytoma, glioma).

c) aerial administration: in disturbances of the respiratory system: rhinitis, sinusitis, pharyngitis, laryngitis, tracheitis, acute and chronic broncho-pneumopathy of phlogistic and/or microbial aetiology due to germs sensitive to these substances (bacteria, fungi, DNA viruses and retroviruses), in particular chronic bronchitis, bronchial asthma, lung diseases due to hypersensitivity, chronic obstructive broncho-pneumopathy, bronchiectasis, bacterial, fungal and viral bronchopulmonitis, particularly in AIDS, alveolar proteinosis, idiopathic interstitial diseases, pulmonary manifestations of leukaemia, tumours of the respiratory system; diseases of the central and peripheral nervous systems of inflammatory, microbial, dystrophic and neoplastic aetiology, in particular anxiety and depression, epilepsy, schizophrenia, movement disturbances (Parkinsons disease, induced by drugs, progressive paralysis), phlogistic and demyelinising degenerative diseases (multiple sclerosis, Alzheimer disease, diseases caused by prions), upper and lower motor neuron diseases (lateral amyotrophic sclerosis, spinal muscular atrophy), use of and dependency from drugs (derivatives of opium, sedatives and hypnotics, cannabis and cocaine), tumour (glioblastoma, astrocytoma, glioma).

The composition of the invention may also be used effectively as an environmental sanitizing agent over the vast antibacterial, anti-mycotic, and anti-mite spectrum, in particular against house mites and for the prevention and treatment of allergic conditions (such as conjunctivitis, rhinitis and bronchial asthma) in patients allergic to mite dust.

It also has a favourable effect in eliminating unpleasant odours and oxidizing substances owing to the presence, in the terpene substance, of one or more of the following functional groups: double-bond cyclic structure or aromatic ring, hydroxyl group. In particular, the carbonyl group reacts with components such as ammonia, amines and mercaptans which are the main sources of unpleasant odours in the food and agricultural industries; some types of terpene aldehydes with the carbonyl group, can react with malodorous aldehydes which may be found in the food industry; the hydroxyl group is a highly polar group and alcohols can therefore react with the aldehydes to form hemiacetals, and can react with carboxylic organic acids (which are one of the causes of unpleasant odours) by esterification reaction.

A further possible use of the composition of the invention is constituted by its use in “aromatology” in order to achieve psycho-physiological effects (a sedative effect on the central and autonomic nervous systems, anxiety and depression) performed on some nerve centres (hypothalamus).

Some specific preferred compositions are listed below with reference to the various forms of preparation and administration of the substances of the invention. It should be noted that, in the compositions which follow, though any ratio between them leads to a synergic effect, the optimum ratio between olibanum derived from Boswellia sacra, myrrh, Dacryoides incense and hyssop is 1:1:0.5:0.5, thus conforming to the optimal proportions between the various terpene fractions indicated above. Naturally, if the olibanum is derived from Boswellia carteri, the proportions will be varied appropriately according to the different terpene content, for example, in accordance with the ratio 1:1:0.5:1.

For Oral Administration

(mean daily dosage for a 60 kg person: 1, 2 times per day)

myrrh 30 mg, free of gum component

Boswellia sacra olibanum 30 mg, free of gum component

Dacryoides incense 15 mg, free of gum component

decumbens hyssop essential oil 15 mg,

polyunsaturated omega-3 fatty acids (EPA+DHA) 900 mg,

the whole mixed 1:10 in an alkaline suspension containing 3% of magnesium hydroxide+3% of aluminium hydroxide, or encapsulated in a shell of gastro-resistant polylactic acid to protect the oral cavity from the unpleasant taste of the omega-3 fatty acids.

The fact that the mixture of terpene-based substances is suspended in an alkaline suspension is particularly important since, in an even moderately acid environment (pH 4-6), these substances give rise to an exothermic reaction, probably owing to the presence of a large number of basic reactive groups, resulting from the considerable quantity of sesquiterpene compounds. For the same reason, with encapsulation in a polylactic acid shell, care must be taken that the shell be gastro-resistant.

For Local Application

a) as ointment applicable directly to the part, or by a spray device, or adsorbed on porous polymers, preferably polylactic acid, once-twice per day until the condition disappears, with the following composition:

Boswellia sacra olibanum (free of gum component): 23%

myrrh (free of gum component): 23%

Dacryoides incense (free of gum component): 11%

decumbens hyssop essential oil: 11%

linseed oil or alcohol: 35%

b) as poultice applicable in the region of the diseased organ or on the nearest skin projection, composed of:

resin in toto with gum component (Boswellia sacra olibanum+myrrh+Dacryoides Incense in balanced ratio): 90-95%

ethyl alcohol: 5-10%

c) as a transdermal patch:

approximately 45 mg/cm² of resin powder, micronized and free of gum component, of Boswellia carteri olibanum+myrrh+Dacryoides incense+decumbens hyssop oil in 1:1:0.5:1 proportions in 10% linseed oil on a patch composed of polylactic acid fibres.

For Aerial Administration

a) in powder with particle size greater than 5 microns In 1:1:0.5 proportions, free of gum component for the treatment of the upper, extrathoracic airways, or with a particle size of from 2 to 5 microns for the treatment of the intrathoracic airways.

b) in 5% to 10% alcoholic solution combined with hyssop essential oil (Hyssopus officinalis var. decumbens) in accordance with the ratios indicated above.

1. A composition of terpene-based substances, comprising a sesquiterpene and/or a triterpene fraction, derived from natural resins in which the sesquiterpene and/or the triterpene fraction are intimately mixed with a gum component of the resins, characterized in that the sesquiterpene and/or triterpene fraction is present in the composition, separated from the gum component.

2. A composition according to claim 1 in which the natural resins are selected from the group constituted by olibanum resin, myrrh resin, and Dacryoides klaineana resin.

3. A composition according to claim 2, comprising the sesquiterpene fraction of Dacryoides klaineana and one or more of the terpene fractions selected from the group consisting of: monoterpene fraction of olibanum, sesquiterpene fraction of olibanum, sesquiterpene fraction of myrrh, triterpene fraction of olibanum, and triterpene fraction of myrrh.

4. A composition according to claim 2, comprising a monoterpene fraction, a sesquiterpene fraction, and a triterpene fraction of the natural resins, characterized in that the ratio between the monoterpene fraction, the sesquiterpene fraction, and the triterpene fraction is 1:1:1, with a variation of ±0.2 for each fraction.

5. A composition according to one or more of the preceding claims, in which the gum component is present in a percentage of less than 2%.

6. A composition according to one or more of the preceding claims in which the terpene-based substances are in powder form and have an average particle-size of less than 5 microns, preferably less than 2 microns, even more preferably about 0.8 microns.

7. A composition according to one or more of the preceding claims in which the terpene-based substances are eluted in vegetable oils or in alcohols with a dilution of between 5% and 40%.

8. A composition according to claim 7 in which the substances are in powder form with a particle size of less than 2 microns, preferably about 0.8 microns, and are eluted in ethanol in a percentage ratio of between 5% and 15%.

9. A composition according to one or more of the preceding claims, further comprising hyssop essential oil.

10. A composition according to claim 9, comprising olibanum, myrrh, Dacryoides klaineana, and hyssop in a ratio by weight of 1:1:0.5:0.5, with a range of variability of ±02 for each of the substances, the olibanum resin being derived from Boswellia sacra plants, or in a ratio of 1:1:0.5:1, with a range of variability of ±0.2 for each of the substances, if the olibanum resin is derived from Boswellia carteri plants.

11. A composition according to claim 9, comprising the sesquiterpene fractions of Dacryoides klaineana, and of myrrh, as well as hyssop essential oil, in substantially equal proportions by weight.

12. A composition according to one or more of claims 9 to 11, wherein the hyssop essential oil is derived from Hyssopus officinalis variety decumbens.

13. A composition according to one or more of claims 9 to 12, wherein the hyssop essential oil is at least partially substituted with an essential oil having a content of trans-linalol-oxide of more than 10% and a content of ketones of less than 1%.

14. A composition according to one or more of the preceding claims, further comprising polyunsaturated omega-3 fatty acids or alpha-linoleic acid.

15. A composition according to claim 14 in which the ratio by weight between the terpene fractions derived from the natural resins and the polyunsaturated omega-3 fatty acids is 1:10±4.

16. A composition according to one or more of the preceding claims in which the terpene-based substances are encapsulated in a water-soluble shell.

17. A composition according to claim 16 in which the shell is based on polylactic acid.

18. A composition according to one or more of claims 1 to 15 in which the terpene-based substances are applied to a transdermal patch with a water-soluble structure.

19. A composition according to claim 18 in which the transdermal patch is based on polylactic acid.

20. A composition according to one or more of the preceding claims in which the terpene-based substances are in an alkaline medium.

21. A method for the separation of terpene fractions from natural resins in which the terpene fractions are intimately mixed with a gum component, characterized in that at least one stripping process is provided for stripping the natural resins in a dry gaseous stream at a temperature such as to cause selective evaporation or liquefaction of the terpene fractions or of the gum component.

22. A method according to claim 21 in which the gaseous stream has an absolute humidity of less than 100 ppm, preferably between 50 and 100 ppm.

23. A method according to claim 21 or claim 22 in which the stripping process is carried out by successive stripping stages, each of the stages having a higher temperature of the dry gaseous stream than that of the previous stage.

24. A method according to claim 23 in which each of the successive stripping stages has a temperature increase of 5° C. relative to the previous stage.

25. A method according to one or more of claims 21 to 24 in which the gaseous stream is supplied to the resin with a flow of between 0.25 and 0.30 m/sec.

26. A method according to one or more of claims 21 to 25 in which the stages have a duration of between 15 and 25 minutes.

27. A method according to one or more of claims 21 to 26 in which the natural resins are selected from the group constituted by olibanum resin, myrrh resin and Dacryoides klaineana resin.

28. A method according to claim 27 in which there is a first stripping stage with a dry gaseous stream at a temperature of between 95° C. and 105° C., a second stage at a temperature of between 130° C. and 138° C., preferably 135° C., a third stage at a temperature of between 138° C. and 142° C., preferably 140.6° C., and a fourth stage at a temperature of between 180° C. and 200° C., preferably 190° C.

29. A method for the preparation of terpene-based compositions comprising the steps of:
providing at least one natural resin comprising a sesquiterpene fraction and/or a triterpene fraction as well as a gum component intimately mixed with the sesquiterpene and/or triterpene fractions, and
combining the fractions with a pharmaceutically acceptable vehicle and/or with other active ingredients,
characterized in that it comprises, prior to the combination step, a step of separation of the sesquiterpene fraction and/or the triterpene fraction from the gum component.

30. A method according to claim 29 in which the natural resins are selected from the group constituted by olibanum resin, myrrh resin, and Dacryoides klaineana resin.

31. A method according to claim 29 or claim 30 in which the separation step is carried out in accordance with the method for the separation of terpene fractions from natural resins of claims 21 to 28.

32. A method according to one or more of claims 29 to 31 in which a step of grinding of at least one of the terpene fractions to a particle size of less than 5 microns, preferably less than 2 microns, even more preferably about 0.8 microns is provided, after the separation step.

33. A method according to claim 32 in which the at least one terpene fraction is eluted in vegetable oils or in alcohols after the grinding.

34. A method according to claim 32 in which the at least one terpene fraction is encapsulated in a water-soluble shell, preferably based on polylactic acid, after the grinding.

35. A method according to claim 32 or claim 33 in which the at least one terpene fraction is applied to a transdermal patch, preferably with a water-soluble structure, after the grinding.

36. A composition which can be produced by the method according to one or more of claims 29 to 35.

37. A composition according to one or more of claims 1 to 20 for use as a medicament.

38. Use of a composition according to one or more of claims 1 to 20 for the preparation of a medical/hygiene product with anti-microbial action.

39. Use of a composition according to claim 10 or claim 11 for the preparation of a medical/hygiene product with anti-Helicobacter pylori and/or anti-Campylobacter jejuni action.

40. Use of a composition according to claims 11 to 13 or claim 15 for the preparation of a medical/hygiene product with anti-mite action, in particular action against house mites and chicken mites.

41. Use of a composition according to one or more of claims 1 to 20 for the preparation of a medical/hygiene product with anti-inflammatory and/or painkilling and/or antiviral and/or anti-tumour and/or anti-malarial action.

42. Use of a composition comprising a monoterpene fraction of olibanum and a monoterpene fraction of hyssop for the production of a medical/hygiene product with anti-microbial action.

43. Use of a composition according to one or more of claims 1 to 20 for the preparation of a medical/hygiene product for the treatment of diseases of the central and peripheral nervous systems of inflammatory, microbial, dystrophic and neoplastic aetiology, in particular anxiety and depression, epilepsy, schizophrenia, movement disturbances (Parkinsons disease, induced by drugs, progressive paralysis); phlogistic and demyelinising degenerative diseases (multiple sclerosis, Alzheimer disease, diseases caused by prions), upper and lower motor neuron diseases (lateral amyotrophic sclerosis, spinal muscular atrophy), use of and dependency from drugs (derivatives of opium, sedatives and hypnotics, cannabis and cocaine), tumour (glioblastoma, astrocytoma, glioma).

44. Use of Dacryoides klaineana or of its extracts for the preparation of a pharmaceutical product with anti-tumour, antiviral, or anti-malarial action.

45. A composition according to one or more of claims 1 to 20 in the form of tablets, pastilles, pills, toothpastes, sprays, sweets, chewing gums, liniments and throat pastilles, toothpastes, mouthwashes, gargles, oils, solutions, emulsions, ointments, salves, creams or preparations for inhalation or nebulization, sprays, aerosols, suppositories, poultices, or preparations for aromatherapy and for cosmetics, as fixatives and fragrance components in soaps, detergents, cosmetics, perfumes, and face powders.

46. A method for the dispersal of terpene-based substances into the atmosphere, comprising the steps of:
pulverizing the substances,
mixing the pulverized substances with a low boiling liquid, and
heating the mixture,
characterized in that the mixture is arranged on a porous material prior to the heating step.

47. A method according to claim 46 in which the porous material has an adsorption capacity of more than 50%, preferably 80%.

48. A method according to claim 46 or claim 47 in which the porous material has an average pore size of between 0.1 and 1 micron.

49. A method according to any one of claims 46, 47 and 48, in which the porous material has an average cell size of between 1 and 10 microns.

50. A method according to one or more of claims 46 to 49 in which the porous material is silica gel, polypropylene, or polylactic acid.

51. A method according to claim 50 in which the porous material is silica gel with an adsorption capacity of 80% and the terpene-based substances are in a ratio of 1:1.25, relative to the silica gel.

52. A method according to one or more of claims 46 to 51 in which the low boiling liquid is ethanol and the mixture is heated to between 80° C. and 90° C.

53. A method according to one or more of claims 46 to 52 in which the terpene-based substances are pulverized to a particle-size of less than 2 microns, preferably about 0.8 microns.

54. A method for the analysis of the anti-microbial activity of volatile fractions of a substance, characterized in that it comprises the steps of:
providing a bacterial suspension of known titre on a sterile glass, drying the glass at a temperature such as not to kill all of the bacteria, arranging the substance on an electro-emanator with controlled temperature heated to an analysis temperature,
putting the glass in contact with the volatile fractions of the substance released by the electro-emanator for a predetermined period of time,
putting the glass in contact with a culture liquid,
filtering through filtering membrane and, after incubation at 37° C. for 24-48 hours, counting the bacterial UFC present on the surface of the filtering membrane.

55. A method according to claim 54 in which the glass is dried at 37° C. for 45-60 minutes in a ventilated oven.

56. A method according to claim 54 or claim 55 in which the substances are terpene-based.
